(19) United States
(12) Patent Application Publication
(10) Pub. No.: US 2006/0015122 A1
(43) Pub. Date: Jan. 19, 2006

(54) Device for guiding a cerclage wire

(76) Inventor: Stephan Rupp, Davos (CH)

Correspondence Address: JONES DAY, 222 EAST 41ST STREET, NEW YORK, NY, 10017-6702, US

(21) Appl. No.: 10/510,939

(22) PCT Filed: Aug. 9, 2002
(86) PCT No.: PCT/CH02/00199

§ 371 Date: Aug. 26, 2005

(51) Int. Cl. A61B 17/58

(52) U.S. Cl. 606/103

(57) ABSTRACT

The invention relates to a device for guiding a cerclage wire around a bone, comprising: A) a longitudinal shaft (6) that can be placed against a bone (2), has a central axis (7), a front end (11) and a central boring (8), which is open at the front end (11) of the shaft (6) while having a central axis (15), and; B) an axially displaceable longitudinal flexible guiding wire (5), which can be extended out of the central boring (8) at the front end (11) of the shaft (6), whereby; C) the central boring (8) at the front end (11) of the shaft (6) has a central axis (15) that is at an angle to the central axis (7) of the shaft (6) whereby causing the guiding wire (5) at the front end (11) of the shaft (6) to extend at an angle to the central axis (7) of the shaft (6).

The invention relates to a device for guiding a cerclage about a bone in accordance with the introductory portion of claim 1.

At the present time, for osteosynthetic interventions, the guiding of a cerclage wire or cable about long tubular bones or at the pelvis is associated with a large detachment of soft parts around the bone. In order to avoid large openings, the cerclage wire must be guided with a special guiding device underneath the tissue around the bone.

A device for guiding a catheter to a particular position, for example, within a blood vessel, by means of a guide wire is known from the EP 0 141 006 of SAKAMOTO. This known device comprises a guide wire for a catheter, which comprises, on the one hand, a distal end section, which is sufficiently flexible so that it can be introduced, for example, into a complex vascular system, and, on the other, a trunk section, which is sufficiently stable to prevent deformation. With regard to guiding the end section about a bone, it is a disadvantage of this known device that the flexible end section is extended linearly coaxially from the catheter, so that a curvature cannot be impressed upon the flexible end section, which is to be introduced, before this end section is guided around a bone.

The invention is to provide a remedy here. It is an object of the invention to create a device for implanting cerclages, by means of which a guide wire can be extended from a stiffer shaft segment telescopically and pre-bent to approximate the curvature of the bone surface, so that the stiffer shaft segment can be placed against the bone and the tip of the guide wire can slip between the bone and the soft parts around the bone.

Pursuant to the invention, this objective is accomplished with a device for guiding a cerclage about a bone, which has the distinguishing features of claim 1.

The inventive device comprises essentially a longitudinal shaft, which can be placed against a bone and is provided with an open central borehole at the front end of the shaft, and a bendable guide wire, which can be extended at the front end of the shaft, so that the free end of the guide wire can be moved axially relative to the front end of the shaft. The central borehole has a central axis, which is angled at the front end of the shaft with respect to the central axis of the shaft. At the same time, the central borehole in this region is shaped so that its central axis is curved or bent at an angle with respect to the central axis of the shaft. By means of this configuration of the central borehole, it is achieved at the guide wire can be extended from the shaft deformed and pre-bent to correspond approximately to the periphery of the bone.

The advantages, achieved by the invention, are to be seen essentially in that, due to the inventive device
only a small incision is required
detachment of the soft parts over a large area is not necessary and
only a slight interference with the blood supply is caused.

In the preferred embodiment of the inventive device, the angle α between the central axis of the shaft and the central axis of the central borehole at the front end of the shaft preferably is between 1° and 90°. The angle selected determines the axial deformation of the guide wire as well as the exit angle at the front end of the shaft with respect to the central axis of the shaft. A better guidance of the guide wire about the bone can be achieved by pre-bending the guide wire in the device.

In a different embodiment of the inventive device, the guide wire is produced from a highly elastic material (such as Nitinol or spring steel).

In a different embodiment of the inventive device, the guide wire is produced from a plastically deformable material, as a result of which a permanent pre-bending can be impressed on the guide wire at the outlet on the shaft.

In yet another embodiment of the inventive device, the latter comprises a headpiece at the front of the guide wire. This headpiece has a borehole with, for example, two constrictions. The different diameters of the borehole and the constrictions permit cerclage wires or cables of different thicknesses to be pressed into the borehole or into one of the constrictions. Furthermore, it can become possible to introduce a cerclage wire or cable into the borehole from a lateral external wall up to the borehole or up to a transverse borehole penetrating into the constrictions and to pass it out of the headpiece once again through the transverse borehole. By these means, it can be achieved that the cerclage wire does not have to be pressed into the borehole or one of the constrictions and, instead, can be fastened to the headpiece by means of a loop.

Various driving mechanisms are possible for axially displacing the guide wire relative to the shaft:
the shaft comprises a front shaft segment and a rear shaft segment, it being possible to telescope the two shaft segments parallel to the central axis of the shaft and relative to one another, so that the guide wire can be extended and retracted by shifting the two shaft segments relative to one another;
the shaft comprises a sliding element, which can be shifted parallel to its central axis and is connected with the guide wire, so that the guide wire can be extended and retracted by axially displacing the sliding element or
the shaft comprises a rack mechanism, which can be moved, for example, by means of a nut in the shaft parallel to the central axis. The guide wire, which is firmly connected with the rack, can be extended or retracted from the shaft by axially moving the rack relative to the shaft.

In a different embodiment of the inventive device, the shaft is angled at its front end, a construction, angled in curved or linear form, also being possible here. The shaft is bent at an angle so that it can be placed better with its front end against the peripherally of the bone.

The guide wire may be cylindrical or prismatic. In order to achieve a high stiffness to counter twisting, the guide wire may have, for example, a rectangular cross section.

In a further embodiment of the inventive device, the guide wire is drilled coaxially and constructed hollow cylindrically or hollow prismatically, so that, for example, a cerclage wire can be accommodated in the cavity of the guide wire and guided, together with the guide wire, around the bone.

Further advantageous embodiments of the invention are characterized in the dependent claims.

The invention and further developments of the invention are explained in even greater detail by means of the partially diagrammatic representations of several examples. In the representations,

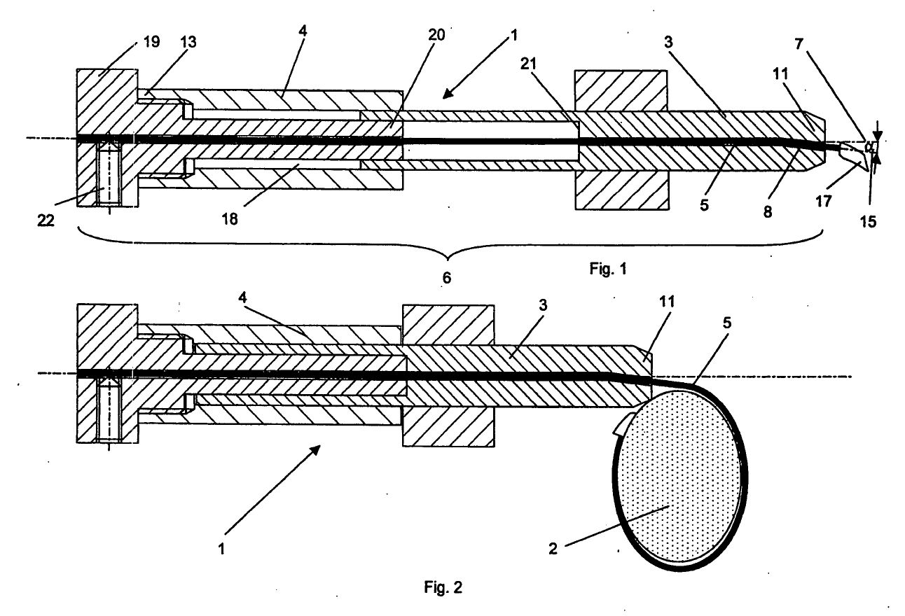

FIG. 1 shows a view of an embodiment of the inventive device with the guide wire retracted;

FIG. 2 shows the embodiment of the inventive device of FIG. 1 with the guide wire extended completely;

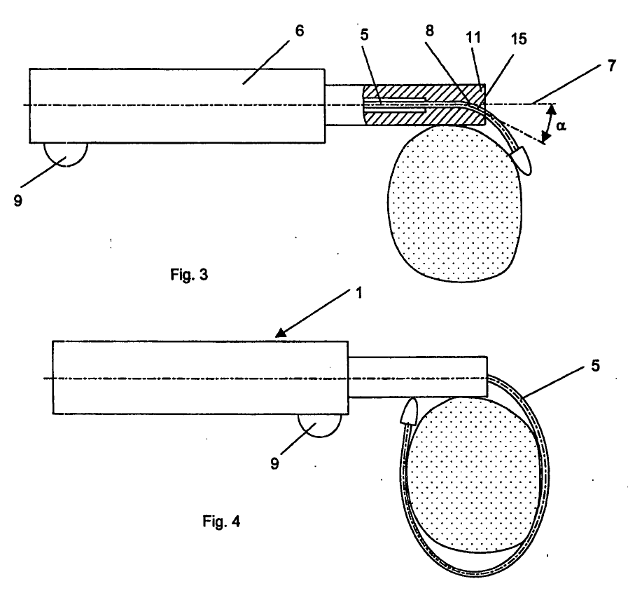

FIG. 3 shows a view of a further embodiment of the inventive device with the guide wire partially extended;

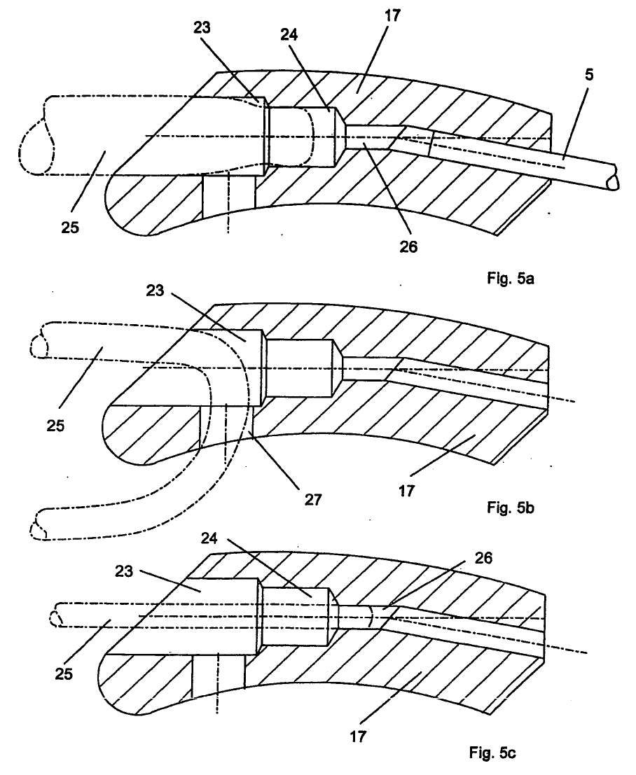

FIG. 4 shows the embodiment of the inventive device of FIG. 3 with the guide wire extended completely;

FIGS. 5a to 5c show a longitudinal section through a head piece of an embodiment of the inventive device;

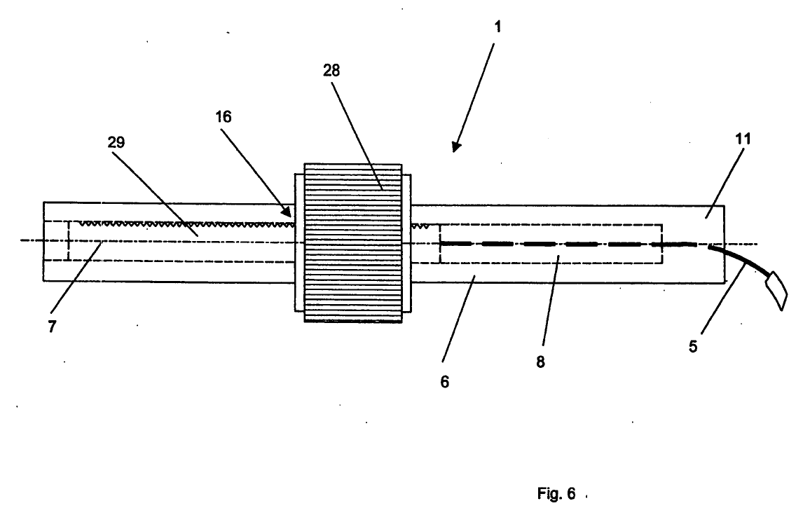

FIG. 6 shows a view of a further embodiment of the inventive device with a rack mechanism and

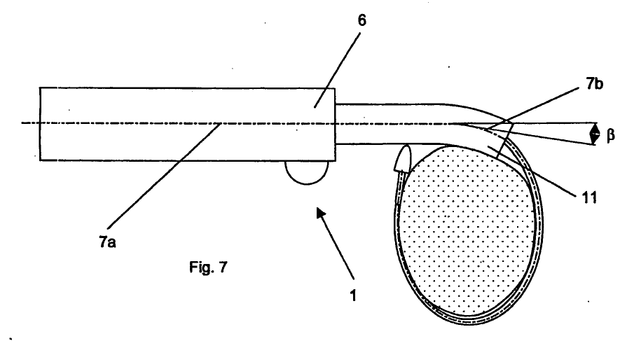

FIG. 7 shows a view of a further embodiment of the inventive device.

In FIG. 1, an embodiment of the inventive device 1 is shown with a shaft 6, having a front shaft segment 3 and a rear shaft segment 4, and a guide wire 5, which can be extended at the front end 11 of the shaft 6. The two shaft segments 3; 4 are configured so that they can be telescoped parallel to the central axis 7. In this embodiment, the front shaft segment 3 can be taken up axially displaceably in the borehole 18 in the rear shaft segment 4, which is coaxial with the central axis 7. At the rear end 13 of the shaft 6, a connecting piece 19 is screwed in, which has a front end 20 protruding coaxially into the borehole 18 in the rear shaft segment 4. When the guide wire 5 is extended completely, the front end 20 of the connecting piece 19 comes up against the shoulder 21 in the central borehole 8 of the front shaft segment 3 (FIG. 2), so that an axial stop for the displaceability of the two shaft segments 3; 4 relative to one another is formed by these means. At the front end 11 of the shaft 6, the central borehole 8 is angled so that the central axis 15 of the central borehole 8 forms an angle α with the central axis 7 of the shaft 6 formed by the 2 shaft segments 3; 4. Furthermore, the guide wire 5 is fixed axially in the connecting piece 19 by a setscrew 22. A headpiece 17, in which a cerclage wire (FIGS. 5a-5c) can be accommodated, is mounted at the front at the guide wire 5.

FIG. 2 shows the device 1 with the guide wire 5 extended completely. For guiding the guide wire 5 around a bone 2, the front end 11 of the shaft 6 is brought into contact with the bone surface and held in this position. If the rear shaft segment 4 is shifted axially relative to the front shaft segment 3, the guide wire 5 is shifted axially along with it and extended at the front end 11 of the shaft 6 at an angle to the central axis 7 of the shaft 6.

FIGS. 3 and 4 show an embodiment of the inventive device, in which the central borehole 8 has a curved central axis 15 at the front end 11 of the shaft 6. At the outlet from the shaft 6, the central axis 15 of the central borehole 8 and the central axis 7 of the shaft 6 once again enclose an angle α. The guide wire 5 is displaced axially by a sliding element 9, which is disposed in the shaft 6 so that it can be displaced axially. The sliding element 9 may be disposed, for example, in a groove (not shown) in the shaft 6. In FIG. 4, the sliding element 9 is in its foremost position, so that the guiding wire 5 can be passed completely around a bone 2.

The device 1 can be inserted through the main incision or through a small incision, which is made especially for this step. The front segment 3 of the shaft 6 is placed against the bone surface and held. Subsequently, the guide wire 5 is extended telescopically by means of a sliding element 9, which can be shifted at the rear shaft segment 4 parallel to the central axis 7 of the shaft 6. At the same time, the free end of the guide wire 5 automatically follows its way around the bone 2. The free end of the guide wire 5 slides between the bone 2 and the soft parts (not shown) and around the bone 2. When the free end of the guide wire 5 has been passed around the bone 2, the cerclage wire (not shown) or the cerclage cable is fastened to the head piece 17 of the guide wire 5 and, by retracting the guide wire 5 by means of the sliding element 9, passed around the bone 2.

An embodiment of the headpiece 17 is shown in FIGS. 5a to 5c. This headpiece 17 is fastened to the front of the guide wire 5 and is passed around a bone 2 (FIG. 2) together with the guide wire 5. The headpiece 17 has a borehole 23, which includes a first and a second constriction 24; 26 in the head piece 17. The dimensions of the constrictions 24; 26 are such, that cerclage wires 25 with different diameters can be pressed, in each case, into one of the constrictions 24; 26. Furthermore, the borehole 23 includes a transverse borehole 27, which passes through the headpiece 17 transversely to the axis of the borehole 23 and from the borehole 23 to the exterior surface of the headpiece 17. With that, a cerclage wire 25 can be introduced into the borehole 23 and passed through the transverse borehole 27, so that a loop can be formed in the cerclage wire 25. The cerclage wire 25 is then fixed by means of the loop to the headpiece 17.

The embodiment of the inventive device 1, shown in FIG. 6, differs from those shown in FIGS. 1 to 4 only in that the guide wire 5 can be moved in the shaft 6 by a rack mechanism 16 parallel to the central axis 7 of the shaft 6 and can be extended and retracted at the front end 11 of the shaft 6 analogously to the embodiments of FIGS. 1 to 4. The rack mechanism 16 comprises essentially a rack 29, which can be displaced in the central borehole 8 parallel to the central axis 7 of the shaft 6 and can be moved by a nut 28, which is connected with the shaft 6 axially fixed and rotatable about the central axis 7. The rack 29 can be moved axially by the internal thread of the nut 28 engaging the teeth of the rack.

FIG. 7 shows an embodiment of the inventive device 1, which differs from the embodiments of FIGS. 1 to 4 and 6 only their in that the shaft 6 is angled or bent at its front end 11, so that the central axis 7b at the front end 11 of the shaft 6 encloses an angle β with the central axis 7a of the remaining length of the shaft 6.

1. A device for guiding a cerclage around a bone, comprising: (a) a longitudinal shaft capable of being placed in contact with a bone, said shaft comprising a central axis, a front end and a central borehole wherein, at the front end of the shaft, the central borehole is angled so that the central axis of the borehole forms an angle to the central axis of the shaft and
(b) an axially displaceable, longitudinally flexible guide wire said guide wire comprising a front end capable of being extended out of the central borehole at the front end of the shaft characterized in that
at the front end of the shaft the guide wire is angled with respect to the central axis of the shaft.

2. The device of claim 1, characterized in that, at the front end of the shaft, the central axis of the central borehole encloses an angle α with the central axis of the shaft, wherein a is from about 1° to about 90°.

3. The device of claim 1, characterized in that the guide wire deformed elastically.

4. The device of claim 1, further comprising a head piece at the front of the guide wire, said head piece comprising a borehole comprising a plurality of constrictions, wherein the diameters of the borehole and of the constrictions are such that different cerclage wires or cables can be introduced into the borehole or into one or more of the constrictions.

5. The device of claim 4, further comprising a transverse borehole in the head piece, said transverse borehole passing through the head piece between the borehole and an exterior wall of the head piece, so that a cerclage wire or cable can be passed in a loop through the borehole and the transverse borehole.

6. The device of claim 1, characterized in that the guide wire is drilled coaxially.

7. The device of claim 1, characterized in that the shaft includes two shaft segments, a front shaft segment and a rear shaft segment and that the front and rear shaft segments may be telescoped parallel to the central axis and relative to one another, so that the guide wire can be extended at the front end of the shaft by retracting one shaft segment into other shaft segment.

8. The device of claim 1, characterized in that the guide wire can be extended from the front end of the shaft by means of a sliding element, which sliding element can be shifted coaxially with the central axis of the shaft.

9. The device of claim 1, characterized in that the guide wire may be extended at the front end of the shaft by means of a rack mechanism.

10. The device of claim 1, characterized in that the shaft is angled at its front end, so that the central axis, at the front end of the shaft, encloses an angle β with the central axis of the remaining length of the shaft.

11. The device of claim 10, characterized in that the angle β is between about 1° and about 90°.

12. The device of claim 5, characterized in that the guide wire is drilled coaxially.

13. The device of claim 6, characterized in that the shaft includes two shaft segments, a front shaft segment and a rear shaft segment and that the front and rear shaft segments may be telescoped parallel to the central axis and relative to one another, so that the guide wire can be extended at the front end of the shaft by retracting one shaft segment into other shaft segment.

14. The device of claim 6, characterized in that the guide wire can be extended from the front end of the shaft by means of a sliding element, which sliding element can be shifted coaxially with the central axis of the shaft.

15. The device of claim 6, characterized in that the guide wire may be extended at the front end of the shaft by means of a rack mechanism.

16. The device of claim 9, characterized in that the shaft is angled at its front end, so that the central axis, at the front end of the shaft, encloses an angle β with the central axis of the remaining length of the shaft.
